(19) United States
(12) Patent Application Publication
(10) Pub. No.: US 2012/0197164 A1
(43) Pub. Date: Aug. 2, 2012

(54) ULTRASOUND COUPLANT

(75) Inventor: Rhianna Moss, Heslington (GB)

(73) Assignee: SMITH & NEPHEW PLC, London (GB)

(21) Appl. No.: 13/390,127

(22) PCT Filed: Aug. 12, 2010
(86) PCT No.: PCT/GB2010/001526

§ 371 Date: Apr. 19, 2012

(30) Foreign Application Priority Data
Aug. 13, 2009 (GB) 0914171.4

(51) Int. Cl. A61N 7/00; G01N 29/00

(52) U.S. Cl. 601/2; 252/408.1

(57) ABSTRACT

The invention relates to the field of absorbent, self-coalescing materials, in particular hydratable polymeric materials, such as cartboxyrmethylchitosan, for use as ultrasound couplants.

### FIELD OF THE INVENTION

The invention relates to the field of absorbent self-coalescing materials, in particular hydratable materials based on polysaccharides, and their use as ultrasound couplants.

### BACKGROUND TO THE INVENTION

Ultrasound, as used for medical applications, utilizes high frequencies, typically between 1 Hz and 20 Hz for imaging and flow measurements. Such frequencies are poorly transmitted by air and require a coupling or conduction medium similar in acoustic properties to tissue, conventionally a viscous gel or fluid, to transfer the acoustic energy between the transducer and the body. This viscosity is particularly advantageous in some medical imaging application, in which the transducer has to be moved across the skin surface.

The treatment of open fractures typically involves the delayed closure of the damaged site for up to 5 days after fracture to allow assessment of the viability of the soft tissue and to reduce the risk of infection. Unfortunately, the physical format of the conventional coupling medium precludes their use in the treatment of open wounds or during the delayed primary closure of open fractures. Concerns include the direct application of a non-sterile medium and ultrasound probe to the wound site and also the difficulty of ensuring complete removal of the viscous material post-treatment. As a result of these concerns these open wounds and fractures have to be closed before ultrasound treatment can begin, which can delay the treatment considerably, for example up to 5 days.

Solid ultrasound coupling agents are known and are primarily used for cushioning. Although these coupling agents minimise some of the concerns associated with the viscous agents, they tend to take the form of pouches which contain a suitable ultrasound transmission medium in a viscous form and as such they have intrinsically dry surfaces which are not favourable for ultrasound probe movement across a surface. Any air captured between the interface between the transducer and the patient will reduced the efficiency of the ultrasound treatment.

Acoustic coupling gels and fluids composed of polysaccharides are known in the art. For example, US 2006/0246111 discloses chitosan-based gels in which chitosan is dissolved in aqueous 2% acetic acid solutions with or without propylene glycol. Whilst such gels are capable of transmitting ultrasound they are highly flowable being characterised by a viscosity in the range of 1,100 cps-5,860 cps (Brookfield LV #2 LVT @3RPM). As such these gels are not suitable for use in open wounds. Whilst ionic-crosslinking, for example with polyvinylpyrrlidone, is suggested as a means of forming gels with varying mechanical properties, including viscosity, such gels are prepared prior to use and hence do not have the ability to absorb any wound exudate in an open wound.

We have identified that a range of polysaccharides, including chitosan and its various salts and derivatives, when applied to open wounds self-coalesce upon hydration caused by the absorption of wound exudate which advantageously results in a substantially solid, pliable gel-like material that is capable of transmitting ultrasound and is therefore ideal as an ultrasound couplant.

There is therefore a need for an ultrasound coupling agent that can be safely applied to open wounds such as open fractures and which also ensures the smooth movement of the ultrasound probe across the treatment surface.

### SUMMARY OF THE INVENTION

According to an aspect of the invention there is provided a fibrous ultrasound couplant material wherein said fibres comprise a polysaccharide and wherein upon hydration of the material said fibres self-coalesce to form an ultrasound transmissible material which is a substantially solid, pliable gel.

According to an aspect of the invention there is provided the use as an ultrasound couplant of a fibrous material, wherein said fibres comprise a polysaccharide.

According to an aspect of the invention there is provided the use of a wound dressing comprising carboxylmethylchitosan fibres as an ultrasound couplant.

According to an aspect of the invention there is provided a method of applying ultrasound to a treatment area, the method comprising the steps of;
i) providing a fibrous material, wherein said fibres comprising a polysaccharide;
ii) hydrating the fibres to form a substantially solid, pliable gel-like material;
iii) contacting an ultrasound transducer with the material formed by the hydration step and transmitting ultrasound through said material to the treatment site.

The term ‘self-coalesce’ is taken to describe the transformation of two or more spatially separated physically homogeneous elements into a single physically homogeneous element or of fusion of previously spatially separated surfaces of the same element.

In embodiments of the invention the material comprises or consists of said fibres.

The material can be a woven or a non-woven fibrous material. The material can be supplied in any convenient form from a manufacturing and/or end-point user point of view. For example the material can be supplied as a fibrous sheet(s) or fibrous pad(s). The material can be used in the geometry as supplied or alternatively it can be readily partitioned into an appropriate geometry for application to a treatment site.

Advantageously the material can be sterilised prior to use to form a barrier between a non-sterile ultrasound probe and the treatment site.

Upon wetting the hydrated fibres of the fibrous material self-coalesce transforming the dry fibrous material into a substantially solid and pliable sheet of gel-like material.

It is envisaged that the material can be wetted either prior to use, by for example a suitable biocompatible solvent, or alternatively during use as a wound dressing, wherein the wound exudate acts as the hydrating fluid. If the material is wetted prior to use, this wetting only needs to occur just prior to use, thereby negating the risk that the material will dry out during storage.

The pliability of this transformed material permits the material to conform to the surfaces that it is placed adjacent to. Advantageously, the surface of the material on which the ultrasound transducer glides is smooth and moist allowing for undisrupted and consistent ultrasound transmission across the material. Advantageously the hydrated material has the integrity to allow it to be picked up in its entirety leaving behind no remnants. This is particularly desirable when the material is used in an open wound.

Suitable polysaccharides can be sourced from marine organisms, terrestrial plants or microbes. Alternatively the polysaccharides can be synthetically derived.

The fibres can comprise or consists of a polysaccharide of the general formula Cₓ(H₂O)_{y}.

In embodiments of the invention the polysaccharide is a linear polysaccharide.

In embodiments of the invention polysaccharide is a long unbranched chain of glucose derivatives.

An example of a suitable polysaccharide which is a long unbranched chain of glucose derivatives is chitosan. This polysaccharide is composed of randomly distributed β-(1-4)-linked D-glucosamine (deacetylated unit) and N-acetyl-D-glucosamine (acetylated unit) and is produced commercially by the deacetylation of chitin (acetylglucosamine—a derivative of glucose). Chitosan's properties allow it to rapidly clot blood, and as such as gained regulatory approval for use in bandages and other hemostatic agents. Additionally chitosan is hypoallergenic, and has natural anti-bacterial properties, further supporting its use in wound dressings. We have identified a further advantageous property of chitosan in that it can transmit ultrasound waves when wet. This wetting causes the chitosan to self-coalesce.

The use of a wound dressing comprising fibres of chitosan, its various salts and derivatives, provides at least the following functionalities when used on a wound: absorptive properties (i.e absorption of wound exudate), hemostatic properties, anti-microbial properties, and ultrasound transmissive properties.

The chitosan, salt or derivative thereof preferably has an average molecular weight (Mw) exceeding 10 kDa (kilodaltons), more preferably exceeding 100 kDa and most preferably exceeding 200 kDa.

Where the polymer is a derivative of chitosan, it is preferably a carboxylated derivative, for example a carboxyalkyl or carboxymethyl derivative. Suitable protocols for achieving carboxymethylation of chitosan are known in the art.

The carboxymethylchitosan preferably has an average molecular weight exceeding 50 kDa, more preferably exceeding 100 kDa, especially exceeding 500 kDa, more especially exceeding 600 kDa and especially 700 kDa or more.

Polysaccharide cellulose derivatives are also envisaged for use in this invention. Suitable examples include, but are in no way limited to; carboxymethylcellulose (CMC), carboxyethylcellulose (CEC), methylhydroxypropylcellulose (MHPC), hydroxyethylceullose (HEC), modified starch and propylene glycol alginate.

An example of a wound dressing comprising carboxymethylcellulose is DURAFIBER® (Smith & Nephew, Inc).

The treatment site for the application of ultrasound can be any site in need thereof, but advantageously this invention enables the application of an ultrasound couplant to an open wound such as an open fracture site.

This invention enables the, to date undiscovered, ultrasound transmissive properties of some of the materials used in conventional wound dressings to be exploited.

According to a further aspect of the invention there is provided an ultrasound couplant comprising a high molecular mass cationic polymer material having a first state which includes at least two separate but adjacent surfaces and a second state in which the polymer consists of a homogeneous body, wherein the material transitions from the first state to the second state upon hydration and wherein upon transition into the second state the material is capable of transmitting ultrasound.

In embodiments of this aspect of the invention the high molecular mass cationic polymer material is chitosan or a salt or derivative thereof, for example carboxymethylchitosan.

According to a further aspect of the invention there is provided materials, methods and uses as substantially herein described with reference to the accompanying Examples and Figures.

### DETAILED DESCRIPTION OF THE INVENTION

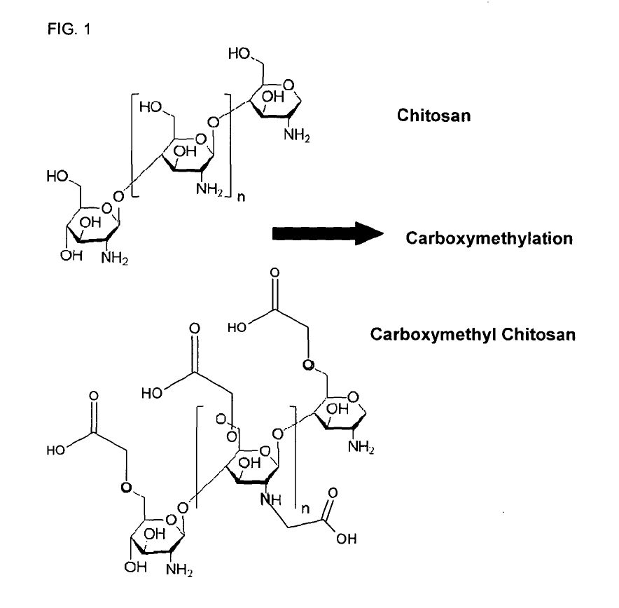

FIG. 1: Modification of chitosan to carboxymethylchitosan (CMCh)

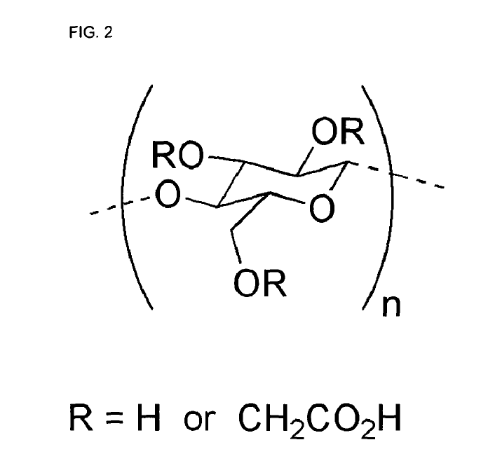

FIG. 2: Chemical structure of carboxymethylcellulose.

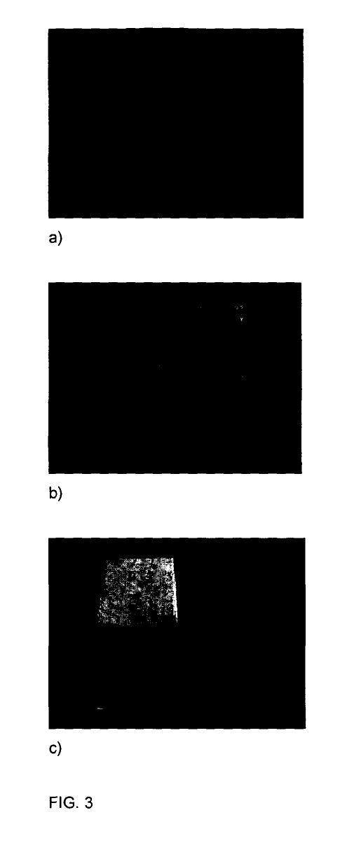

FIG. 3a) non-woven CMCh, b) non-woven CMCh half wetted, c) non-woven CMCh dry and wet.

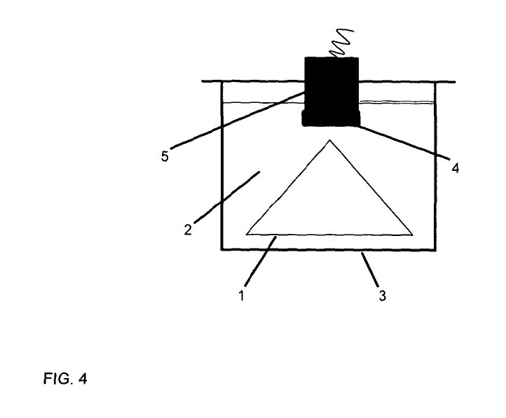

FIG. 4: Experimental set-up used to record ultrasound transmission

### EXAMPLES

### Example 1

### Generation of Self-Coalescing Carboxymethylchitosan Fibres

A) Synthesis

Immediately prior to reaction, sodium chloroacetate (1.75 g) was dissolved in 4% aqueous sodium hydroxide solution (7 ml). This solution was added to isopropanol (45 ml) and shaken vigorously, resulting in a turbid suspension. This mixture was added to a vessel containing chitosan fibres (1.50 g), the container sealed and rolled at approximately 60 rpm for 18 hours.

B) Washing Steps

Step B1) After step A, the fibres were removed from the now clear reaction solvent and transferred to a vessel containing 99:1 ethanol:water (200 ml). The material was disturbed every 15 minutes for 1 hour, after which time the material was removed and physically dried by the application of hand pressure between several layers of absorbent material. Following gross drying, the material was vacuum dried at ambient temperature overnight.

Step B2) After step A, the fibres were removed from the now clear reaction solvent and transferred to a vessel containing 60:40 ethanol:water (200 ml). The material was disturbed every 15 minutes for 1 hour, after which time the material was removed and transferred to a second vessel containing 90:10 ethanol:water (200 ml). The material was disturbed every 15 minutes for 1 hour, after which time the material was removed and physically dried by the application of hand pressure between several layers of absorbent material. Following gross drying, the material was vacuum dried at ambient temperature overnight.

### Example 2

### Generation of Self-Coalescing Carboxymethylchitosan Fibres (Scale-Up)

Immediately prior to reaction, sodium chloroacetate (96.8 g) was dissolved in 4% aqueous sodium hydroxide solution (387 ml). This solution was added to isopropanol (2490 ml) and shaken vigorously, resulting in a turbid suspension. This mixture was added to a vessel containing chitosan fibres (83.0 g), the container sealed and rolled at approximately 60 rpm for 18 hours. After this time, the fibres were removed from the now clear reaction solvent and transferred to a vessel containing 99:1 ethanol:water (2000 ml). The material was disturbed every 15 minutes for 1 hour, after which time the material was removed and physically dried by the application of hand pressure between several layers of absorbent material. Following gross drying, the material was vacuum dried at ambient temperature overnight.

### Example 3

### Fibrous Pad Formation

The carboxymethylchitosan fibres formed in Example 1 are processed into a non-woven felt.

A variety of additives such as antibacterials and antimicrobials can then be added to the carboxymethylchitosan non-woven.

Typical Densities of the Non-Woven Felt:

Areal density 30-200 g/m² (OMT), 100-200 g/m²

Volume density 0.05-01 g/cm³ for a loft (thickness of the non-woven) of 2 mm.

In an alternative embodiment of the invention chitosan fibres are processed into a non-woven felt pad and then chemically functionalised into a carboxymethylchitosan non-woven. This carboxymethylchitosan non-woven is dried.

### Example 4

### Gamma Irradation of Self-Coalescing Carboxymethylchitosan Fibres

The material resulting from Example 1, step B2 was packaged in gas-permeable sterilisation pouches and sterilised by gamma irradiation at 30-40 kGy. The molecular weight of the material pre-and post-sterilisation was determined by gel permeation chromatography. The molecular weight prior to sterilisation was about Mw 700 kDa (as determined by gel permeation chromatography); the molecular weight post-sterilisation was between about Mw 100-150 kDa (gamma radiation sterilisation). The molecular weight change in the material was such that the physical properties of the material were not significantly altered by sterilisation.

### Example 5

### Ethylene Oxide Sterilisation of Self-Coalescing Carboxymethylchitosan Fibres

The material resulting from Example 1, step B2 was packaged in gas-permeable sterilisation pouches and sterilised by ethylene oxide treatment. The molecular weight of the material pre-and post-sterilisation was determined by gel permeation chromatography. The molecular weight prior to sterilisation was approximately Mw 700 kDa (as determined by gel permeation chromatography); the molecular weight post-sterilisation was between about Mw 500 kDa-600 KDa. The molecular weight change in the material was such that the physical properties of the material were not significantly altered by sterilisation.

### Example 6

### Self-Coalescence Upon Hydration

FIG. 3a shows a modified chitosan pad

FIG. 3b shows a modified chitosan pad that has been part immersed in fluid

FIG. 3c compares a dry chitosan pad with a gelled chitosan pad

### Example 7

### Ultrasound Transmission Through the Chitosan Pad

Ultrasound transmission through the chitosan pad was recorded using an Ohmic power balance and standard EXOGEN® (Smith & Nephew, Inc( transducer (see FIG. 4). The power balance has a light weight cone [1], mounted vertex up, instead of a pan. The cone is submerged in degassed, deionised water [2] in a rubber-lined tank [3]. The material to be tested [4] is placed on the end of the transducer [5] (held in place with cling-film) and placed directly over the vortex of the cone. The force produced by the ultrasound beam, dependent on the transmission media, is recorded and converted directly into units of power (mW).

The average power transmission recorded through the chitosan pad was 82 mW. The average power recorded for a liquid transmission gel using the same method is 109 mW.

The higher the value, the better the ultrasound transmission.

1. (canceled)

2. A method of applying ultrasound to a treatment area, the method comprising the steps of;
i) providing a fibrous material comprising fibers, wherein the fibers comprise a polysaccharide;
ii) hydrating the fibers to form a substantially solid, pliable gel-like material; and
iii) contacting an ultrasound transducer with the material formed by the hydrating; and
iv) transmitting ultrasound using the ultrasound transducer through the material.

3. The method according to claim 2 wherein the polysaccharide is a linear polysaccharide.

4. The method according to claim 3, wherein the linear polysaccharide is chitosan or a salt or derivative thereof.

5. The method according to claim 4, wherein the chitosan is carboxymethylchitosan.

6. The method according to claim 3, wherein the linear polysaccharide is cellulose or a salt or derivative thereof.

7. The method according to claim 6, wherein the cellulose derivative is selected from a group consisting of carboxymethylcellulose (CMC), carboxyethylcellulose (CEC), methylhydroxypropylcellulose (MHPC), hydroxyethylceullose (HEC), modified starch and propylene glycol alginate.

8-10. (canceled)

11. A fibrous ultrasound couplant material comprising:
fibers constructed of a polysaccharide, wherein upon hydration of the material the fibers self-coalesce to form an ultrasound transmissible material that is a substantially solid, pliable gel.

12. The couplant material according to claim 11, wherein the polysaccharide includes a linear polysaccharide.

13. The couplant material according to claim 12, wherein the linear polysaccharide includes chitosan or a salt or derivative thereof.

14. The couplant material according to claim 13, wherein the chitosan includes carboxymethylchitosan.

15. The couplant material according to claim 12, wherein the linear polysaccharide includes cellulose or a salt or derivative thereof.

16. The couplant material according to claim 15, wherein the cellulose derivative is selected from a group consisting of carboxymethylcellulose (CMC), carboxyethylcellulose (CEC), methylhydroxypropylcellulose (MHPC), hydroxyethylceullose (HEC), modified starch and propylene glycol alginate.

17. A system comprising:
an ultrasound couplant that is configured to be placed in contact with a treatment site, the ultrasound couplant comprising a high molecular mass cationic polymer material having a first state that includes at least two separate but adjacent surfaces and a second state in which the material consists of a homogeneous body, wherein the material transitions from the first state to the second state upon hydration, and wherein upon transitioning into the second state the material is capable of transmitting ultrasound; and
an ultrasound transducer that is configured to couple with the ultrasound couplant when the material is in the second state and transmit ultrasound through the ultrasound couplant to the treatment site.

18. The system according to claim 17, wherein the ultrasound couplant comprises fibers constructed of a polysaccharide when the material is in the first state, and wherein the ultrasound couplant comprises a substantially solid, pliable gel when the material is in the second state, the substantially solid, pliable gel being formed due to self-coalescing of the fibers upon hydration of the material.

19. The system according to claim 18, wherein the polysaccharide includes a linear polysaccharide.

20. The system according to claim 19, wherein the linear polysaccharide includes chitosan or a salt or derivative thereof

21. The system according to claim 20, wherein the chitosan includes carboxymethylchitosan.

22. The system according to claim 19, wherein the linear polysaccharide includes cellulose or a salt or derivative thereof.

23. The system according to claim 22, wherein the cellulose derivative is selected from a group consisting of carboxymethylcellulose (CMC), carboxyethylcellulose (CEC), methylhydroxypropylcellulose (MHPC), hydroxyethylceullose (HEC), modified starch and propylene glycol alginate.

24. The system according to claim 17, wherein the ultrasound couplant forms at least a part of a wound dressing.
